(19) United States
(12) Patent Application Publication
(10) Pub. No.: US 2002/0065259 A1
(43) Pub. Date: May 30, 2002

(54) Glucocorticoid blocking agents for increasing blood-brain barrier permeability

(76) Inventors: Alan F. Schatzberg, Los Altos, CA (US); Joseph K. Belanoff, Woodside, CA (US); Steven Lindley, Redwood City, CA (US)

Correspondence Address: Bret E. Field, Bozicevic, Field and Francis LLP, Suite 200, 200 Middlefield Road, Menlo Park, CA, 94025, US

(21) Appl. No.: 09/942,531

(22) Filed: Aug. 29, 2001

Related U.S. Application Data
(60) Provisional application No. 60/229,278, filed on Aug. 30, 2000.

(51) Int. Cl. A61K 31/57

(52) U.S. Cl. 514/179

(57) ABSTRACT

Glucocorticoid blockers, including glucocorticoid receptor antagonists, are effective to prevent glucocorticoid-induced decrease in permeability of the blood-brain barrier and to increase the permeability of the blood-brain barrier. Administration of glucocorticoid blockers, including glucocorticoid receptor antagonists, concomitant with administration of drugs for treating diseases of the central nervous system increases delivery of such drugs into the central nervous system.

### CROSS REFERENCE TO RELATED APPLICATIONS

Pursuant to 35 U.S.C. § 119(e), this application claims priority to the filing date of the U.S. Provisional Patent Application Ser. No. 60/229,278, filed Aug. 30, 2000, the disclosure of which is herein incorporated by reference.

### INTRODUCTION

1. Field of the Invention

This invention relates to methods and formulations for increasing the permeability of the blood-brain barrier. In particular, this invention relates to methods of using glucocorticoid blockers, such as glucocorticoid receptor antagonists, to increase the permeability of the blood-brain barrier and to pharmaceutical compositions containing glucocorticoid receptor antagonists.

2. Description of Related Art

Steroid hormones are well known to have significant effects on animal cells. Corticosteroids are steroid hormones released by the adrenal glands. The most significant human adrenal corticosteroids are cortisol, corticosterone and aldosterone. Based on their observed effects on carbohydrate, mineral and water metabolism, these compounds have been divided into two classes: the mineralocorticoids, affecting mineral and water metabolism, such as aldosterone; and the glucocorticoids, affecting carbohydrate metabolism, such as corticosterone and cortisol (hydrocortisone, 17-hydroxycorticosterone). Corticosterone can act as both a glucocorticoid and as a mineralocorticoid.

Corticosteroids produce cellular effects following binding to receptors located in the cell membrane. Ligand-bound receptors are subsequently internalized and migrate to the nucleus of the cell, where they act on the nuclear material to alter gene expression in the cell. Two general classes of corticosteroid receptors are now recognized, the mineralocorticoid receptors (also termed type I, or MR) and the glucocorticoid receptors (also termed type II, or GR, or cortisol receptors). In addition, it is well known that there are also other steroid receptors which may be present on some animal cells. An example of another steroid hormone receptor is the progesterone receptor.

Mineralocorticoid receptors (MRs) bind corticosterone with high affinity, and bind glucocorticoids with a ten-fold higher affinity than glucocorticoid receptors (GRs) bind glucocorticoids. Thus, the activation of the two classes of receptors may differ depending on the corticosteroid concentration. Blood levels of the glucocorticoid cortisol vary over a wide range during the day. In general, normal cortisol concentrations in the blood range from about 0.5 nM to about 50 nM; however, in response to stress, cortisol concentration may exceed 100 nM.

Glucocorticoid blockers are agents that block or reduce the effects of glucocorticoids. Such interference with glucocorticoid action may, for example, be due to interference with binding of glucocorticoid agonists to glucocorticoid receptors (GR), or to interference with the action of agonist-bound GR at the cell nucleus, or to interference with expression or processing of gene products induced by the action of agonist-bound GR at the nucleus. Glucocorticoid receptor antagonists (GR antagonists) are compounds which inhibit the effect of the native ligand or of glucocorticoid agonists on GR. One mode of action of GR antagonists is to inhibit the binding of GR ligands to GR. A discussion of glucocorticoid antagonists may be found in Agarwal et al. “Glucocorticoid antagonists”, FEBS Lett., 217:221-226 (1987). An example of a GR antagonist is mifepristone, (11β,17β)-11-[4-(dimethylamino)phenyl]-17-hydroxy-17-(1-propynyl)estra-4,9-dien-3-one, also known as RU-486 or RU-38486. See U.S. Pat. No. 4,368,085. Mifepristone binds specifically to GR with high affinity (Kd≦10⁻⁹ M). This is an affinity about 18 times that of the affinity of cortisol for GR. GR antagonists may be steroids, such as mifepristone, or non-steroids.

Examples of other steroidal GR antagonists include androgen-type steroid compounds as described in U.S. Pat. No. 5,929,058, and the compounds disclosed in U.S. Pat. Nos. 4,296,206; 4,386,085; 4,447,424; 4,477,445; 4,519,946; 4,540,686; 4,547,493; 4,634,695; 4,634,696; 4,753,932; 4,774,236; 4,808,710; 4,814,327; 4,829,060; 4,861,763; 4,912,097; 4,921,638; 4,943,566; 4,954,490; 4,978,657; 5,006,518; 5,043,332; 5,064,822; 5,073,548; 5,089,488; 5,089,635; 5,093,507; 5,095,010; 5,095,129; 5,132,299; 5,166,146; 5,166,199; 5,173,405; 5,276,023; 5,380,839; 5,348,729; 5,426,102; 5,439,913; 5,616,458, and 5,696,127. Such steroidal GR antagonists include cortexolone, dexamethasoneoxetanone, 19-nordeoxycorticosterone, 19-norprogesterone, cortisol-21-mesylate dexamethasone-21-mesylate, 11β-(4-dimethylaminoethoxyphenyl)-17α-propynyl-17β-hydroxy -4,9-estradien-3-one (RU009), and 17β-hydroxy-17β-19-(4-methylphenyl)androsta -4,9(11)-dien-3-one (RU044).

Examples of other non-steroidal GR antagonists include ketoconazole, clotrimazole; N-(triphenylmethyl)imidazole; N-([2-fluoro-9-phenyl]fluorenyl)imidazole; N-([2-pyridyl]diphenylmethyl)imidazole; N-(2-[4,4′,4″-trichlorotrityl]oxyethyl)morpholine; 1-(2[4,4′,4″-trichlorotrityl]oxyethyl)-4-(2-hydroxyethyl)piperazine dimaleate; N-([4,4′,4″]-trichlorotrityl)imidazole; 9-(3-mercapto-1,2,4-triazolyl)-9-phenyl-2,7-difluorofluorenone; 1-(2-chlorotrityl)-3,5-dimethylpyrazole; 4-(morpholinomethyl)-A-(2-pyridyl)benzhydrol; 5-(5-methoxy-2-(N-methylcarbamoyl)-phenyl)dibenzosuberol; N-(2-chlorotrityl)-L-prolinol acetate; 1-(2-chlorotrityl)-2-methylimidazole; 1-(2-chlorotrityl)-1,2,4-triazole; 1,S-bis(4,4′,4″ -trichlorotrityl)-1,2,4-triazole-3-thiol; and N-((2,6-dichloro-3-methylphenyl)diphenyl)methylimidazole (see U.S. Pat. No. 6,051,573); and the GR antagonist compounds disclosed in U.S. Pat. No. 5,696,127; the compounds disclosed in PCT International Application No. WO 96/19458, which describes non-steroidal compounds which are high-affinity, highly selective antagonists for steroid receptors, such as 6-substituted-1,2-dihydro-N-protected-quinolines; and some κ opioid ligands, such as the κ opioid compounds dynorphin-1,13-diamide, U50,488 (trans-(1R,2R)-3,4-dichloro-N-methyl -N-[2-(1-pyrrolidinyl)cyclohexyl]benzeneacetamide), bremazocine and ethylketocyclazocine; and the non-specific opioid receptor ligand, naloxone, as disclosed in Evans et al., Endocrin., 141:2294-2300 (2000).

The disclosures of all patents, patent applications, and other documents cited in this application are incorporated by reference.

It has long been recognized that the central nervous system (CNS) is a privileged compartment within an animal, and that transport between the blood and the CNS is less rapid, more difficult and more closely regulated than transport between the blood and other body compartments. The “blood-brain barrier” (“BBB”) is the term used to describe this functional barrier between the central nervous system and the blood of an animal.

The BBB is affected by corticosteroids. For example, corticosteroids are reported to decrease BBB permeability (Hedley-Whyte et al., Ann. Neurol. 19:373-377 (1986); Neuwelt et al., J. Neurosurg. 72:123-126 (1990); Paul et al., Int. J. Immunopharm. 17:497503 (1995); Ziylan et al., J. Neurochem. 51:1338-1342 (1988), Ziylan et al., J. Neurochem. 52:684-689 (1989) and Ziylan et al., Mol. and Chem. Neuropath. 20:203-218 (1993)). Adrenalectomy, which lowers corticosteroid levels, increases BBB permeability (Brown et al., Tox. and Appl. Pharm. 150:158-165 (1988) and Long et al., Science 227:1580-1583 (1985)).

It is well established that stress, whether physical stress such as disease, injury or exercise, or psychological stress, such as anxiety, depression, or fear, leads to increased corticosteroid levels.

Although it is believed that the BBB serves a protective function under normal conditions by protecting the CNS from exposure to potentially toxic compounds, in CNS disease the BBB may thwart therapeutic efforts by hindering the entry of therapeutic compounds into the CNS. For example, although many bacterial and fungal infections may be readily treated where the site of the infection is outside the CNS, such infections in the CNS are often very dangerous and very difficult to treat due to the inability to deliver effective doses of drugs to the site of the infection. Similarly, the action of the BBB makes treatment of cancer of the brain more difficult than treatment of cancers located outside the CNS. Even where it may be possible to deliver an effective dose of drug into the CNS by administering very large amounts of drug outside of the CNS, the drug levels outside the CNS (such as in the blood) are then often so high as to reach toxic levels deleterious to the kidneys, liver, and other vital organs. Accordingly, there is need in the art for methods to improve the delivery of compounds into the CNS.

### SUMMARY OF THE INVENTION

In a first aspect, this invention provides a method of increasing the permeability of the blood-brain barrier in an animal, comprising administering to the animal a blood-brain barrier permeability-increasing effective amount of a glucocorticoid blocker.

In a second aspect, this invention provides a method of increasing the permeability of the blood-brain barrier in an animal, comprising administering to the animal a blood-brain barrier permeability-increasing effective amount of a glucocorticoid receptor antagonist.

In a third aspect, this invention provides a method of preventing a decrease in the permeability of the blood-brain barrier in an animal induced by increased cortisol levels in the animal, comprising administering to the animal a blood-brain barrier permeability-decrease-preventing effective amount of a glucocorticoid receptor antagonist.

In a fourth aspect, this invention provides a method of treating an animal having a disease capable of treatment by increasing the permeability of the blood-brain barrier in the animal, comprising administering to the animal a therapeutically effective amount of a glucocorticoid receptor antagonist.

In a fifth aspect, this invention provides a method of enhancing the delivery of a drug to the central nervous system of an animal, comprising concomitantly administering to the animal with that drug a blood-brain barrier permeability-increasing effective amount of a glucocorticoid receptor antagonist.

In a sixth aspect, this invention provides a method of treating an animal having a disease of its central nervous system capable of treatment by a drug administered to its central nervous system, comprising concomitantly administering to the animal a therapeutically effective amount of said drug and a blood-brain barrier permeability-increasing effective amount of a glucocorticoid receptor antagonist.

In a seventh aspect, this invention provides a pharmaceutical composition for treating a disease of the central nervous system, comprising: a therapeutically effective amount of a drug useful for treating the disease, a blood-brain barrier permeability-increasing effective amount of a glucocorticoid receptor antagonist, and a pharmaceutically acceptable excipient.

In an eighth aspect, this invention provides a kit for the treatment of a disease of the central nervous system, comprising: a therapeutically effective amount of a drug useful for treating the disease, a blood-brain barrier permeability-increasing effective amount of a glucocorticoid receptor antagonist, and instructions for the concomitant administration of the drug and the glucocorticoid receptor antagonist.

### DETAILED DESCRIPTION OF THE PREFERRED EMBODIMENTS

Definitions

“Animal” includes humans and non-human mammals, such as companion animals (cats, dogs, and the like) and farm animals (cattle, horses, sheep, goats, swine, and the like).

“Disease” includes any unhealthy condition of an animal, including particularly tumors, especially tumors of the internal organs, and parasitic, bacterial, fungal, and viral infections.

“CNS disease” means any unhealthy condition of the central nervous system (CNS) of an animal. An unhealthy condition may be the result of the presence of undesirable organisms, such as bacteria, fungi, viruses, or other disease-causing organisms, or may be the result of the presence of undesirable cells, such as malignant cells, or excessive white blood cells, or other cells whose presence causes a disease condition, or may be the result of the presence of undesirable materials, such as toxins, metals, metabolites, peptides, plaques, or other materials, or may be a condition of unknown origin, such as psychosis, schizophrenia, depression, or other psychiatric condition.

The term “glucocorticoid receptor” (abbreviated “GR”) denotes a molecule or molecules that bind glucocorticoids with high affinity; in particular, GR refers to the type II corticosteroid receptor.

The term “glucocorticoid blocker” denotes a molecule or molecules that block or reduce the effects of glucocorticoids. Any compound effective to antagonize glucocorticoid action is a glucocorticoid blocker.

The term “glucocorticoid receptor antagonist” (abbreviated “GR antagonist”) denotes compounds which inhibit the effect of the native ligand or of GR agonists on GR. GR antagonists are glucocorticoid blockers.

“Concomitant administration” of a drug with a glucocorticoid blocker means administration of the drug and the glucocorticoid blocker at such times that the drug is present in the blood at such a level that the drug can reach a therapeutically effective level in the CNS when the BBB is lowered by a BBB-lowering amount of a glucocorticoid blocker. Such concomitant administration may involve concurrent (i.e. at the same time), prior or subsequent administration of the drug with respect to the administration of a glucocorticoid blocker, depending on the onsets of action and half-lives of the drug and glucocorticoid blocker chosen. A person of ordinary skill in the art, having knowledge of the drugs and glucocorticoid blockers, would have no difficulty determining the appropriate timing, sequence and dosages of administration for particular drugs and glucocorticoid blockers.

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

“Pharmaceutically acceptable salts and esters” means salts and esters that are pharmaceutically acceptable and have the desired pharmacological properties. Such salts include salts that may be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g. ethanolamine, diethanolamine, triethanolamine, tromethamine, N-methylglucamine, and the like. Such salts also include acid addition salts formed with inorganic acids (e.g. hydrochloric and hydrobromic acids) and organic acids (e.g. acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds, e.g. C₁₋₆ alkyl esters. When there are two acidic groups present, a pharmaceutically acceptable salt or ester may be a mono-acid-mono-salt or ester or a di-salt or ester; and similarly where there are more than two acidic groups present, some or all of such groups can be salified or esterified. Compounds named in this invention may be present in unsalified or unesterified form, or in salified and/or esterified form, and the naming of such compounds is intended to include both the original (unsalified and unesterified) compound and its pharmaceutically acceptable salts and esters. Also, certain compounds named in this invention may be present in more than one stereoisomeric form, and the naming of such compounds is intended to include all single stereoisomers and all mixtures (whether racemic or otherwise) of such stereoisomers.

A “therapeutically effective amount” means the amount that, when administered to an animal for treating a disease, is sufficient to effect treatment for that disease. In the case of a drug for treating a disease of the CNS that is concomitantly administered with a BBB-permeability-increasing effective amount of a glucocorticoid blocker, the therapeutically effective amount of the drug when concomitantly administered with the glucocorticoid blocker will be lower than the therapeutically effective amount of the drug when not concomitantly administered with the glucocorticoid blocker. Thus, a drug concomitantly administered with a glucocorticoid blocker may be effective in treating a CNS disease at a blood level that would be ineffective in the absence of the effects of the glucocorticoid blocker.

“Treating” or “treatment” of a disease includes preventing the disease from occurring in an animal that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease).

The Method of the Invention

Antagonism to glucocorticoid activity and to the action of glucocorticoid receptors is effective to block the decrease in BBB permeability observed following increases in cortisol levels, as occurs during stress. In addition, inhibition of the action of glucocorticoid receptors is effective to increase the permeability of the BBB. Increased BBB permeability is effective to increase the delivery of therapeutic drugs into the CNS.

Accordingly, administration of glucocorticoid blockers is effective to increase BBB permeability. Administration of glucocorticoid blockers concomitant with the administration of drugs directed towards treating a condition of the CNS increases delivery of the drug into the CNS thereby increasing the effectiveness of the drug treatment and making such treatment possible at lower drug dosages than would be possible in the absence of glucocorticoid blockers.

Steroidal Anti-Glucocorticoids as Glucocorticoid Blockers

In one embodiment of the invention, steroidal glucocorticoid blockers are administered to increase BBB permeability. Steroidal antiglucocorticoids, such as steroidal GR antagonists, can be obtained by modification of the basic structure of glucocorticoid agonists, i.e., varied forms of the steroid backbone. The structure of cortisol can be modified in a variety of ways. The two most commonly known classes of structural modifications of the cortisol steroid backbone to create glucocorticoid blockers include modifications of the 11β-hydroxy group and modification of the 17β side chain. (Lefebvre et al., J. Steroid Biochem. 33:557-563 (1989)).

Removal or Substitution of the 11-β Hydroxy Group

In another embodiment of the invention, glucocorticoid agonists with modified steroidal backbones comprising removal or substitution of the 11β-hydroxy group are administered. This class includes natural antiglucocorticoids, including cortexolone, progesterone and testosterone derivatives, and synthetic compositions, such as mifepristone (Lefebvre, et al., cited above). Preferred embodiments of the invention include all 11β-aryl steroid backbone derivatives because these compounds are devoid of progesterone receptor (PR) binding activity (Agarwal, cited above). Another preferred embodiment comprises an 11β-[4-(dimethylamino)phenyl] steroid backbone derivative, i.e., mifepristone, which is both an effective anti -glucocorticoid and anti-progesterone agent. These compositions act as reversibly-binding steroid receptor antagonists. For example, when bound to a 11-[4(dimethylamino)phenyl] steroid, the steroid receptor is maintained in a conformation that cannot bind its natural ligand, such as cortisol in the case of GR (Cadepond, et al., Ann. Rev. Med. 48:129 (1997)).

Synthetic 11-β phenyl-aminodimethyl steroids include mifepristone, also known as RU486, or 17-β-hydroxy-11-β-(4-dimethyl-aminophenyl) 17-β-(1 -propynyl)estra-4,9-dien-3-one). It has been shown to be both a powerful progesterone receptor antagonist and a powerful GR antagonist. Other 11-β phenyl-aminodimethyl steroids shown to have GR antagonist effects include RU009 (RU39.009), 11-p-(4-dimethyl-aminoethoxyphenyl)-17α-(propynyl-17β-hydroxy-4,9-estradien-3-one) (see Bocquel (1993) J. Steroid Biochem. Molec. Biol. 45:205-215). Another GR antagonist related to RU486 is RU044 (RU43.044) 17-β-hydroxy-17-α-19-(4-methyl-phenyl)-androsta-4,9(11)-dien-3-one) (Bocquel (1993)supra). See also Teutsch (1981) Steroids 38:651-665: U.S. Pat. Nos. 4,386,085 and 4,912,097.

Some GR antagonist compounds containing the basic glucocorticoid steroid structure are irreversible anti-glucocorticoids. Such compounds include α-keto-methanesulfonate derivatives of cortisol, including cortisol-21-mesylate (4-pregnene-11-β,17-α,21-triol-3,20-dione-21-methane-sulfonate and dexamethasone-21-mesylate (16-methyl-9α-fluoro -1,4-pregnadiene-11β,17-α,21-triol-3,20-dione-21-methane-sulfonate). See Simons (1986) J. Steroid Biochem. 24:25-32 (1986); Mercier (1986) J. Steroid Biochem. 25:11-20; U.S. Pat. No. 4,296,206.

Modification of the 17-β Side Chain Group

Steroidal antiglucocorticoids which can be obtained by various structural modifications of the 17-β side chain are also used in the methods of the invention. This class includes synthetic antiglucocorticoids such as dexamethasone-oxetanone, various 17, 21-acetonide derivatives and 17-β-carboxamide derivatives of dexamethasone (Lefebvre, et al. (1989) supra; Rousseau (1979) Nature 279:158-160).

Other Steroid Backbone Modifications

GR antagonists used in the various embodiments of the invention include any steroid backbone modification which effects a biological response resulting from a GR-agonist interaction. Steroid backbone antagonists can be any natural or synthetic variation of cortisol, such as adrenal steroids missing the C-19 methyl group, such as 19nordeoxycorticosterone and 19-norprogesterone (Wynne (1980) Endocrinology 107:1278-1280).

In general, the 11-β side chain substituent, and particularly the size of that substituent, can play a key role in determining the extent of a steroid's anti glucocorticoid activity. Substitutions in the A ring of the steroid backbone can also be important. 17-hydroxypropenyl side chains generally decrease antiglucocorticoidal activity in comparison to 17-propynyl side chain containing compounds.

Non-Steroidal Anti-Glucocorticoids as Glucocorticoid Blockers

Non-steroidal glucocorticoid antagonists are also used in the methods of the invention to increase BBB permeability or to prevent a decrease in BBB due to glucocorticoid action. These include synthetic mimetics and analogs of proteins, including partially peptidic, pseudopeptidic and non-peptidic molecular entities. For example, oligomeric peptidomimetics useful in the invention include (α-β-unsaturated) peptidosulfonamides, N-substituted glycine derivatives, oligo carbamates, oligo urea peptidomimetics, hydrazinopeptides, oligosulfones and the like (de Bont (1996) Bioorganic & Medicinal Chem. 4:667-672). The creation and simultaneous screening of large libraries of synthetic molecules can be carried out using well-known techniques in combinatorial chemistry, for example, see van Breemen (1997) Anal Chem 69:2159-2164; Lam (1997) Anticancer Drug Des 12:145-167 (1997). Peptidomimetics specific for GR can be designed using computer programs in conjunction with combinatorial chemistry, (combinatorial library) screening approaches (Murray (1995) J. Computer-Aided Molec. Design 9:381-395); Bohm (1996) J. Computer-Aided Molec. Design 10:265-272). Such “rational drug design” can help develop peptide isomerics and conformers including cycloisomers, retro-inverso isomers, retro isomers and the like (as discussed in Chorev (1995) TibTech 13:438-445).

Identifying Glucocorticoid Blockers

Because any glucocorticoid blocker can be used for increasing the permeability of the BBB or preventing or reducing glucocorticoid-induced decreases in BBB permeability in the methods of the invention, in addition to the compounds and compositions described above additional useful glucocorticoid blockers can be determined by the skilled artisan. A variety of such routine, well-known methods can be used and are described in the scientific and patent literature. They include in vitro and in vivo assays for the identification of additional glucocorticoid blockers.

Presently Preferred Compounds

While the broadest definition of the invention is set out in the Summary of the Invention, and it is herein taught that any glucocorticoid blocker, such as, for example, a compound effective to antagonize glucocorticoid binding at a GR, is suitable for the practice of the invention, including all glucocorticoid blockers named herein, both supra and infra, certain GR antagonist compounds as taught in this invention are presently preferred.

Suitable GR antagonist compounds include mifepristone, cortexolone, dexamethasone-oxetanone, 19-nordeoxycorticosterone, 19-norprogesterone, cortisol-21mesylate; dexamethasone-21-mesylate, 11β-(4-dimethylaminoethoxyphenyl)-17α-propynyl -17-hydroxy-4,9-estradien-3-one (RU009), and 17α-hydroxy-17α-19-(4-methylphenyl)androsta -4,9(11)-dien-3-one (RU044).

Suitable other steroidal GR antagonists include androgen-type steroid compounds as described in U.S. Pat. No. 5,929,058, and the compounds disclosed in U.S. Pat. Nos. 4,296,206; 4,386,085; 4,447,424; 4,477,445; 4,519,946; 4,540,686; 4,547,493; 4,634,695; 4,634,696; 4,753,932; 4,774,236; 4,808,710; 4,814,327; 4,829,060; 4,861,763; 4,912,097; 4,921,638; 4,943,566; 4,954,490; 4,978,657; 5,006,518; 5,043,332; 5,064,822; 5,073,548; 5,089,488; 5,089,635; 5,093,507; 5,095,010; 5,095,129; 5,132,299; 5,166,146; 5,166,199; 5,173,405; 5,276,023; 5,380,839; 5,348,729; 5,426,102; 5,439,913; 5,616,458, and 5,696,127.

Suitable non-steroidal GR antagonists include ketoconazole, clotrimazole; N-(triphenylmethyl)imidazole; N-([2-fluoro-9-phenyl]fluorenyl)imidazole; N-([2-pyridyl]diphenylmethyl)imidazole; N-(2-[4,4′,4″-trichlorotrityl]oxyethyl)morpholine; 1-(2[4,4′,4″-trichlorotrityl]oxyethyl)-4-(2-hydroxyethyl)piperazine dimaleate; N-([4,4′,4″]trichlorotrityl)imidazole; 9-(3-mercapto-1,2,4-triazolyl)-9-phenyl-2,7-difluorofluorenone; 1-(2-chlorotrityl)-3,5-dimethylpyrazole; 4-(morpholinomethyl)-A-(2-pyridyl)benzhydrol; 5-(5-methoxy-2-(N-methylcarbamoyl)-phenyl)dibenzosuberol; N-(2-chlorotrityl)-L-prolinol acetate; 1-(2-chlorotrityl)-2-methylimidazole; 1-(2-chlorotrityl)-1,2,4-triazole; 1, S-bis(4,4′,4″-trichlorotrityl)-1,2,4-triazole-3-thiol; and N-((2,6-dichloro-3-methylphenyl)diphenyl)methylimidazole (see U.S. Pat. No. 6,051,573); and the GR antagonist compounds disclosed in U.S. Pat. No. 5,696,127; the compounds disclosed in PCT International Application No. WO 96/19458, which describes non-steroidal compounds which are high-affinity, highly selective antagonists for steroid receptors, such as 6-substituted-1,2-dihydro-N-protected-quinolines; and some κ opioid ligands, such as the κ opioid compounds dynorphin-1,13-diamide, U50,488 (trans-(1R,2R)-3,4-dichloro-Nmethyl-N-[2-(1-pyrrolidinyl)cyclohexyl]benzeneacetamide), bremazocine and ethylketocyclazocine; and the non-specific opioid receptor ligand, naloxone, as disclosed in Evans et al., Endocrin., 141:2294-2300 (2000).

Presently, the preferred glucocorticoid blocker is the GR antagonist mifepristone.

Pharmaceutical Compositions and Administration

In general, glucocorticoid blockers suitable for use in the practice of this invention will be administered in therapeutically effective amounts by any of the usual modes known in the art, either singly or in combination with at least one other compound of this invention and/or at least one other conventional therapeutic agent for the disease being treated. A therapeutically effective amount may vary widely depending on the disease, its severity, the age and relative health of the animal being treated, the potency of the compound(s), and other factors. Therapeutically effective amounts of glucocorticoid blockers suitable for practice of the method of the invention may range from about 0.5 to about 25 milligrams per kilogram (mg/kg). A person of ordinary skill in the art will be able without undue experimentation, having regard to that skill and this disclosure, to determine a therapeutically effective amount of a particular glucocorticoid blocker compound for practice of this invention.

In general, glucocorticoid blocker compounds may be administered as pharmaceutical compositions by one of the following routes: oral, topical, systemic (e.g. transdermal, intranasal, or by suppository), or parenteral (e.g. intramuscular, subcutaneous, or intravenous injection). Compositions may take the form of tablets, pills, capsules, semisolids, powders, sustained release formulations, solutions, suspensions, elixirs, aerosols, or any other appropriate compositions; and comprise at least one compound of this invention in combination with at least one pharmaceutically acceptable excipient. Suitable excipients are well known to persons of ordinary skill in the art, and they, and the methods of formulating the compositions, may be found in such standard references as Alfonso A R: Remington's Pharmaceutical Sciences, 17th ed., Mack Publishing Company, Easton Pa., 1985. Suitable liquid carriers, especially for injectable solutions, include water, aqueous saline solution, aqueous dextrose solution, and glycols.

Glucocorticoid blocker pharmaceutical formulations can be prepared according to any method known to the art for the manufacture of pharmaceuticals. Such drugs can contain sweetening agents, flavoring agents, coloring agents and preserving agents. Any glucocorticoid blocker formulation can be admixtured with nontoxic pharmaceutically acceptable excipients which are suitable for manufacture.

Typically, glucocorticoid blocker compounds suitable for use in the practice of this invention will be administered orally. The amount of a compound of this invention in the composition may vary widely depending on the type of composition, size of a unit dosage, kind of excipients, and other factors well known to those of ordinary skill in the art. In general, the final composition may comprise from 0.000001 percent by weight (%w) to 10%w of the glucocorticoid blocker compounds, preferably 0.00001%w to 1%w, with the remainder being the excipient or excipients. For example, the GR antagonist mifepristone is given orally in tablet form, with dosages in the range of between about 0.5 and 25 mg/kg, more preferably between about 0.75 mg/kg and 15 mg/kg, most preferably about 10 mg/kg.

Pharmaceutical formulations for oral administration can be formulated using pharmaceutically acceptable carriers well known in the art in dosages suitable for oral administration. Such carriers enable the pharmaceutical formulations to be formulated in unit dosage forms as tablets, pills, powder, dragees, capsules, liquids, lozenges, gels, syrups, slurries, suspensions, etc. suitable for ingestion by the patient. Pharmaceutical preparations for oral use can be obtained through combination of glucocorticoid blocker compounds with a solid excipient, optionally grinding a resulting mixture, and processing the mixture of granules, after adding suitable additional compounds, if desired, to obtain tablets or dragee cores. Suitable solid excipients are carbohydrate or protein fillers and include, but are not limited to sugars, including lactose, sucrose, mannitol, or sorbitol; starch from corn, wheat, rice, potato, or other plants; cellulose such as methyl cellulose, hydroxypropylmethyl-cellulose or sodium carboxymethylcellulose; and gums including arabic and tragacanth; as well as proteins such as gelatin and collagen. If desired, disintegrating or solubilizing agents may be added, such as the cross-linked polyvinyl pyrrolidone, agar, alginic acid, or a salt thereof, such as sodium alginate.

A pharmaceutical composition of the invention may optionally contain, in addition to a glucocorticoid blocker compound, at least one other therapeutic agent useful in the treatment of a disease or condition of the CNS. Such other compounds may be of any class of drug or pharmaceutical agent, including but not limited to antibiotics, anti-parasitic agents, antifungal agents, anti-viral agents and anti-tumor agents. When administered with anti-parasitic, anti-bacterial, anti-fungal, anti-tumor, anti-viral agents, and the like, glucocorticoid blocker compounds may be administered by any method and route of administration suitable to the treatment of the disease, typically as pharmaceutical compositions.

Glucocorticoid Blocker Dosage Regimens

The methods of the invention increase BBB permeability and/or prevent or reduce glucocorticoid-induced decreases in BBB permeability. The amount of glucocorticoid blocker adequate to accomplish this is defined as a “therapeutically effective dose.” The dosage schedule and amounts effective for this use, i.e. the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration. The dosage regimen may also take into consideration the pharmacokinetics, i.e., the glucocorticoid blockers' rate of absorption, bioavailability, metabolism, clearance, and the like (see, for example, Hidalgo-Aragones (1996) J. Steroid Biochem. Mol. Biol. 58:611-617; (Droning; (1996) Pharmazie 51:337-341; Fotherby (1996) Contraception 54:59-69; Johnson (1995) T Pharm. Sci. 84:1144-1146; Rohatagi (1995) Pharmazie 50:610-613; Brophy (1983) Eur. J. Clin. Pharmacol. 24:103-108; Remington's Pharmaceutical Science, 15th ed., Mack Publishing Company, Easton, Pa. (1980)).

The state of the art allows the clinician to determine the dosage regimen for each individual patient, glucocorticoid blocker, and disease or condition treated. Glucocorticoid blocker compounds suitable for use in the practice of this invention may be administered as single or multiple dosages. The example provided below for mifepristone can be used to guide the determination of the dosage regimen, including dosing schedule and dosage levels, of any glucocorticoid blocker administered when practicing the methods of the invention.

For example, a typical preferred pharmaceutical formulation for oral administration of mifepristone would be about 5 to 15 mg/kg of body weight per patient per day, more preferably between about 8 to about 12 mg/kg of body weight per patient per day, most preferably 10 mg/kg of body weight per patient per day, although dosages of between about 0.5 to about 25 mg/kg of body weight per day maybe used in the practice of the invention. Even wider range of dosages may be utilized in some instances, such as, for example, topical administration, or where the drug is administered to an anatomically secluded site, such as the cerebral spinal fluid (CSF) space, in contrast to oral administration, or administration into the blood stream, into a body cavity, or into the lumen of an organ. Actual methods for preparing parenterally administrable glucocorticoid blockers formulations will be known or apparent to those skilled in the art and are described in more detail in such publications as Remington's Pharmaceutical Science, 15th ed., Mack Publishing Company, Easton, Pa. (1980). At the preferred dosage of about 8 to 20 mg/kg of body weight per patient per day, administration can continue for a period of about 4 days. In an alternative dosing regimen, mifepristone may be administered in a daily amount of between about 300 mg/day to about 800 mg/day, more preferably about 600 mg/day.

Glucocorticoid Blocker Kits

After a pharmaceutical comprising a glucocorticoid blocker has been formulated in a suitable carrier, it can be placed in an appropriate container and labeled for treatment of an indicated disease. Optionally, another pharmaceutical comprising at least one other therapeutic agent useful in the treatment of a disease of the CNS may be placed in the container as well, and labeled for treatment of the indicated disease. Alternatively, a single pharmaceutical comprising a glucocorticoid blocker and at least one other therapeutic agent useful in the treatment of a disease of the CNS can be placed in an appropriate container and labeled for treatment of an indicated disease. For administration of pharmaceuticals comprising glucocorticoid blockers and of pharmaceuticals comprising, in a single pharmaceutical, glucocorticoid blockers and at least one other therapeutic agent useful in the treatment of a disease of the CNS, such labeling would include, for example, instructions concerning the amount, frequency and method of administration. Similarly, for administration of multiple pharmaceuticals provided in the container, such labeling would include, for example, instructions concerning the amount, frequency and method of administration of each pharmaceutical.

In one embodiment, the invention provides for a kit for the treatment of a disease of the CNS, which includes a glucocorticoid blocker and instructional materials teaching the indications, dosage, and schedule of administration of the glucocorticoid blocker. When mifepristone is the glucocorticoid blocker provided in the kit, the instructional material indicates that the glucocorticoid blocker can be used in a daily amount of about 8 to 12 mg/kg of body weight per day, and the administration of the glucocorticoid blocker continues for a period of about four days.

In the light of the foregoing, and of the examples presented below, it will be understood by one of ordinary skill in the art that administration of a glucocorticoid blocker may be for a longer or a shorter period of time than four days, and, if concomitantly administered with another drug, that the glucocorticoid blocker may be given at the same time, or may be administered beginning minutes, hours, or days before or after administration of the other drug depending on the characteristics of the particular compounds and the status of the patient.

### EXAMPLES

### Example 1

### Corticosteroid Administration Decreases BBB Permeability

Adrenalectomized rats (male Sprague Dawley 175-200 grams) were implanted with drug-release pellets (Innovative Research of America, Sarasota, Fla.) and maintained, 3 rats per cage, under controlled temperature and lighting (12 hours light/12 hours dark) with food and water ad libitum. The implanted pellets contained either 100 mg corticosterone (for haloperidol experiments), 50 mg corticosterone (for clozapine experiments) or placebo. Two hours prior to sacrifice the animals were injected with either haloperidol (1 mg/kg s.c; RBI Natick, Mass.) or an equivalent volume of vehicle (0.3% tartaric acid, pH 5.3) or with either clozapine (15 mg/kg s.c.; RBI, Natick, Mass.) or vehicle (0.9% saline plus 0.8% acetic acid).

Animals were sacrificed by decapitation during the first four hours of the light cycle, blood collected and brains removed and frozen on dry ice and stored at −80° C. Corticosterone was measured in plasma by radioimmunoassay (ICN Biochem, Costa Mesa, Calif.) to confirm adrenalectomy and corticosterone replacement. Frozen brains were sliced into 250 μm sections with a cryostat. The medial prefrontal cortex (AP 13.7 to 12.2 mm) was dissected with a scalpel, the striatum (AP 10.7 to 9.7 mm) removed with stainless steel cannulae from frozen slices and the core and shell of the nucleus accumbens (AP 10.7 to 9.7 mm) was removed. Brain regions were dissected within 24-72 hours of slicing. Tissue was placed in 0.1 M perchloric acid with 0.1 mM EDTA and stored for no longer than 2 weeks at −80° C.

Dopamine metabolites homovanillic acid (HVA) and dihydroxyphenylacetic acid (DOPAC) were measured in the brains of the experimental animals. Samples were thawed, homogenized by sonication in 0.1 M perchloric acid/0.1 mM EDTA, and centrifuged for 2 minutes. Tissue pellets were dissolved in 1.0 N NaOH for protein determination (Bio-Rad, Richmond, Calif.). Cortical supernatants were filtered through a 0.45 μm filter and 5-80 μl of supernatant was injected directly onto a C18 reverse phase analytical column (5 μm, 250×4.6 mm; Biophase ODS, BAS, West Lafayette, Ind.) protected by a precolumn cartridge (5 μm, 30×4.6 mm, BAS) as described with modification (Lindley et al. Proc. Soc. Exp. Biol. Med. 188:282-286 (1988)). DOPAC and HVA were detected using an electrochemical detector. For cortical regions, the conditioning electrode was set at +0.35 V and the dual analytical electrode was set at +0.02 V and −0.35 V, respectively (ESA, Bedford, Mass.). For other regions a single analytical electrode set at +0.72 V was used (BAS, West Lafayette, Ind.). Brain clozapine levels were analyzed by National Medical Services, Inc. (Willow Grove, PN) while brain and plasma haloperidol and reduced haloperidol and plasma clozapine levels were analyzed by Analytical Psychopharmacology Laboratories (Nathan Kline Institute, Orangeburg, N.Y.), both by gas chromotography.

Consistent with prior work demonstrating that both haloperidol and clozapine increase dopamine utilization in the brain, measured levels of HVA and DOPAC were elevated in the brains of vehicle-treated animals. However, the effects of haloperidol and clozapine on dopamine metabolite levels were smaller in corticosterone-treated animals than in animals receiving vehicle pellets. In addition, corticosterone-treatment also significantly decreased brain concentrations of haloperidol, the reduced form of haloperidol, and clozapine without decreasing plasma reduced haloperidol or plasma clozapine levels. Thus, corticosterone inhibits both haloperidol-induced and clozapine-induced increases in dopamine metabolite levels in the brain.

### Example 2

### Glucocorticoid Blocker-induced Increase in Permeability of the BBB

Rats (male Sprague Dawley 175-200 grams) are maintained, 3 rats per cage, under controlled temperature and lighting (12 hours light/12 hours dark) with food and water ad libitum. One week prior to sacrifice, rats are given mifepristone (200 mg) or placebo 10day sustained-release pellet. Two hours prior to sacrifice the animals are injected with either haloperidol (1 mg/kg s.c; RBI Natick, Mass.) or an equivalent volume of vehicle (0.3% tartaric acid, pH 5.3) or with either clozapine (15 mg/kg s.c.; RBI, Natick, Mass.) or vehicle (0.9% saline plus 0.8% acetic acid).

Animal sacrifice is by decapitation during the first four hours of the light cycle. Blood is collected and brains removed and frozen on dry ice for storage at −80° C. Frozen brains are sliced into 250 μm sections with a cryostat. The medial prefrontal cortex (AP 13.7 to 12.2 mm) is dissected with a scalpel, the striatum (AP 10.7 to 9.7 mm) is removed with stainless steel cannulae from frozen slices and the core and shell of the nucleus accumbens (AP 10.7 to 9.7 mm) is removed. Brain regions are dissected within 24-72 hours of slicing. Tissue is placed in 0.1 M perchloric acid with 0.1 mM EDTA for storage for no longer than 2 weeks at −80° C.

Dopamine metabolites homovanillic acid (HVA) and dihydroxyphenylacetic acid (DOPAC) are measured in the brains of the experimental animals. Samples are thawed and are homogenized by sonication in 0.1 M perchloric acid/0.1 mM EDTA, and centrifuged for 2 minutes. Tissue pellets are dissolved in 1.0 N NaOH for protein determination (BioRad, Richmond, Calif.). Cortical supernatants are filtered through a 0.45 μm filter and 5-80 μl of supernatant is injected directly onto a C18 reverse phase analytical column (5 μm, 250×4.6 mm; Biophase ODS, BAS, West Lafayette, Ind.) protected by a precolumn cartridge (5 μm, 30×4.6 mm, BAS) as described with modification (Lindley et al. Proc. Soc. Exp. Biol. Med. 188:282-286 (1988)). DOPAC and HVA were detected using an electrochemical detector. For cortical regions, the conditioning electrode is set at +0.35 V and the dual analytical electrode is set at +0.02 V and −0.35 V, respectively (ESA, Bedford, Mass.). For other regions a single analytical electrode set at +0.72 V is used (BAS, West Lafayette, Ind.). Brain clozapine levels are analyzed by National Medical Services, Inc. (Willow Grove, PN) while brain and plasma haloperidol and reduced haloperidol and plasma clozapine levels are analyzed by Analytical Psychopharmacology Laboratories (Nathan Kline Institute, Orangeburg, N.Y.), both by gas chromatography.

Levels of haloperidol, clozapine and the dopamine metabolites homovanillic acid (HVA) and dihydroxyphenylacetic acid (DOPAC) are measured in the brains of the experimental animals. Measured levels of HVA and DOPAC are elevated in the brains of vehicle-treated animals. The increase in dopamine metabolite levels following haloperidol and clozapine treatment, as well as the brain concentrations of haloperidol and clozapine, are greater in mifepristone-treated animals than in animals receiving placebo. This demonstrates glucocorticoid blocker-induced increases in haloperidol and clozapine levels in the brain and potentiation of haloperidol and clozapine-induced increases in dopamine metabolite levels in the brain, consistent with an increase in BBB permeability due to mifepristone.

### Example 3

### Glucocorticoid Blocker-induced Increase in Permeability of the BBB and Resulting Increase in Delivery of Amphotericin B

Amphotericin B is a polyene antibiotic with potent antifungal activity.

Rats (male Sprague Dawley 175-200 grams) are maintained, 3 rats per cage, under controlled temperature and lighting (12 hours light/12 hours dark) with food and water ad libitum. One week prior to sacrifice, rats are given mifepristone (200 mg) or placebo 10day sustained release pellet. Two hours prior to sacrifice the animals are injected with either Amphotericin B (0.1 mg i.v.; Sigma Chemical Co., (800) 325-3010) or an equivalent volume of vehicle (0.1% DMSO in saline, pH 11).

Animal sacrifice is by decapitation during the first four hours of the light cycle. Blood is collected and brains removed and frozen on dry ice for storage at −80° C. Amphotericin B concentration is measured in the brains of the experimental animals. The Amphotericin B concentration is greater in the brains of mifepristone-treated animals than in placebo-treated animals. This result demonstrates a glucocorticoid blocker-induced increase in Amphotericin B delivery to the brain, consistent with an increase in BBB permeability due to mifepristone.

### Example 4

### Glucocorticoid Blocker-induced Increase in Permeability of the BBB and Resulting Increase in Delivery of Ampicillin

Ampicillin (D[−]-α-Aminobenzylpenicillin) is a potent antibacterial agent structurally related to penicillin.

Rats (male Sprague Dawley 175-200 grams) are maintained, 3 rats per cage, under controlled temperature and lighting (12 hours light/12 hours dark) with food and water ad libitum. One week prior to sacrifice, rats are given mifepristone (200 mg) or placebo 10-day sustained release pellet. Two hours prior to sacrifice the animals are injected with either ampicillin (1.5 mg i.v.; Sigma Chemical Co., (800)325-3010) or an equivalent volume of vehicle (0.9% sodium chloride colution).

Animal sacrifice is by decapitation during the first four hours of the light cycle. Blood is collected and brains removed and frozen on dry ice for storage at −80° C. Ampicillin concentration is measured in the brains of the experimental animals. The ampicillin concentration is greater in the brains of mifepristone-treated animals than in placebo-treated animals. This result demonstrates a glucocorticoid blocker-induced increase in ampicillin delivery to the brain, consistent with an increase in BBB permeability due to mifepristone.

### Example 5

### Glucocorticoid Blocker-induced Increase in Permeability of the BBB and Resulting Increase in Delivery of Methotrexate

Methotrexate (N-[4-[[)2,4-Diamino-6-pteridinyl-)methylamino]benzoyl]-L-glutamic acid) is a folic acid antagonist that is a potent cancer chemotherapy agent.

Rats (male Sprague Dawley 175-200 grams) are maintained, 3 rats per cage, under controlled temperature and lighting (12 hours light/12 hours dark) with food and water ad libitum. One week prior to sacrifice, rats are given mifepristone (200 mg) or placebo 10 day sustained-release pellet. Two hours prior to sacrifice the animals are injected with either methotrexate (0.5 mg i.v; Sigma Chemical Co., (800)325-3010) or an equivalent volume of vehicle (saline, pH 9).

Animal sacrifice is by decapitation during the first four hours of the light cycle. Blood is collected and brains removed and frozen on dry ice for storage at −80° C. Methotrexate concentration is measured in the brains of the experimental animals. The methotrexate concentration is greater in the brains of mifepristone-treated animals than in placebo-treated animals. This result demonstrates a glucocorticoid blocker-induced increase in methotrexate delivery to the brain, consistent with an increase in BBB permeability due to mifepristone.

### Example 6

### Glucocorticoid Blocker-induced Increase in Permeability of the BBB and Resulting Increase in Delivery of Adriamycin

Adriamycin ((8S-cis)-10-(3-Amino-2,3,6-Trideoxy-alpha-L-Lyxo-Hexopyranosyl)Oxy-7,8,9,10-Tetrahydro-6,8,11-Trihydroxy-8-(Hydroxyacetyl)-1-Methoxy-5,12-Naphthacenedione, also known as doxorubicin) is a potent cancer chemotherapy agent.

Rats (male Sprague Dawley 175-200 grams) are maintained, 3 rats per cage, under controlled temperature and lighting (12 hours light/12 hours dark) with food and water ad libitum. Four hours prior to sacrifice rats are injected with mifepristone (dissolved in benzyl benzoate-sesame oil(1:4) with slight warming; dosage 2 mg s.c.) or placebo. Two hours prior to sacrifice the animals are injected with either adriamycin (0.5 mg i.v; “doxorubicin hydrochloride,” Sigma Chemical Co., (800)325-3010) or an equivalent volume of vehicle (saline, pH 9).

Animal sacrifice is by decapitation during the first four hours of the light cycle. Blood is collected and brains removed and frozen on dry ice for storage at −80° C. Adriamycin concentration is measured in the brains of the experimental animals. The adriamycin concentration is greater in the brains of mifepristone-treated animals than in placebo-treated animals. This result demonstrates a glucocorticoid blocker-induced increase in adriamycin delivery to the brain, consistent with an increase in BBB permeability due to mifepristone.

While this invention has been described in conjunction with specific embodiments and examples, it will be apparent to a person of ordinary skill in the art, having regard to this disclosure, that equivalents of the specifically disclosed materials and techniques will also be applicable to this invention; and such equivalents are intended to be included within the following claims.

What is claimed is:

1. A method of increasing the permeability of the blood-brain barrier in an animal, comprising administering to the animal a blood-brain barrier permeability-increasing effective amount of a glucocorticoid blocker.

2. The method of claim 1, wherein the glucocorticoid blocker comprises a glucocorticoid receptor antagonist.

3. A method of preventing a decrease in the permeability of the blood-brain barrier in an animal induced by increased corticosteroid levels in the animal, comprising administering to the animal a blood-brain barrier permeability-decrease-preventing effective amount of a glucocorticoid blocker.

4. The method of claim 3, wherein the glucocorticoid blocker comprises a glucocorticoid receptor antagonist.

5. A method of treating an animal having a disease capable of treatment by increasing the permeability of the blood-brain barrier in the animal, comprising administering to the animal a therapeutically effective amount of a glucocorticoid blocker.

6. The method of claim 5, wherein the glucocorticoid blocker comprises a glucocorticoid receptor antagonist.

7. A method of enhancing the delivery of a drug to the central nervous system of an animal, comprising concomitantly administering to the animal with that drug a blood-brain barrier permeability-increasing effective amount of a glucocorticoid blocker.

8. The method of claim 7, wherein the glucocorticoid blocker comprises a glucocorticoid receptor antagonist.

9. A method of treating an animal having a disease of its central nervous system capable of treatment by a drug administered to its central nervous system, comprising concomitantly administering to the animal a therapeutically effective amount of said drug and a blood-brain barrier permeability-increasing effective amount of a glucocorticoid blocker.

10. The method of claim 9, wherein the glucocorticoid blocker comprises a glucocorticoid receptor antagonist.

11. A pharmaceutical composition for treating a disease of the central nervous system, comprising:
a therapeutically effective amount of a drug useful for treating the disease,
a blood-brain barrier permeability-increasing effective amount of a glucocorticoid blocker, and
a pharmaceutically acceptable excipient.

12. A pharmaceutical composition for treating a disease of the central nervous system, comprising:
a therapeutically effective amount of a drug useful for treating the disease,
a blood-brain barrier permeability-increasing effective amount of a glucocorticoid receptor antagonist, and
a pharmaceutically acceptable excipient.

13. A kit for the treatment of a disease of the central nervous system, comprising:
a therapeutically effective amount of a drug useful for treating the disease,
a blood-brain barrier permeability-increasing effective amount of a glucocorticoid blocker, and
instructions for the concomitant administration of the drug and the glucocorticoid receptor antagonist.

14. A kit for the treatment of a disease of the central nervous system, comprising:
a therapeutically effective amount of a drug useful for treating the disease,
a blood-brain barrier permeability-increasing effective amount of a glucocorticoid receptor antagonist, and
instructions for the concomitant administration of the drug and the glucocorticoid receptor antagonist.

15. The method of claim 2, where the glucocorticoid receptor antagonist is a steroidal glucocorticoid receptor antagonist.

16. The method of claim 15 where the steroidal glucocorticoid receptor antagonist is selected from the group consisting of mifepristone, cortexolone, dexamethasone-oxetanone, 19-nordeoxycorticosterone, 19-norprogesterone, cortisol-21-mesylate; dexamethasone-21-mesylate, 11(-(4-dimethylaminoethoxyphenyl)-17(-propynyl-17(-hydroxy-4,9-estradien-3one, and 17(-hydroxy-17(-19-(4-methylphenyl)androsta-4,9(11)-dien-3-one.

17. The method of claim 16 where the steroidal glucocorticoid receptor antagonist is mifepristone.

18. The method of claim 2, where the glucocorticoid receptor antagonist is a non-steroidal glucocorticoid receptor antagonist.

19. The method of claim 18 where the non-steroidal glucocorticoid receptor antagonist is selected from the group consisting of ketoconazole, clotrimazole NB, (triphenylmethyl)imidazole, N-([2-fluoro-9-phenyl]fluorenyl)imidazole, N-([2-pyridyl]diphenylmethyl)imidazole, N-(2-[4,4′,4″-trichlorotrityl]oxyethyl)morpholine, 1-(2[4,4′,4″-trichlorotrityl]oxyethyl)-4-(2-hydroxyethyl)piperazine dimaleate, N-([4,4′,4″]-trichlorotrityl)imidazole, 9-(3-mercapto-1,2,4-triazolyl)-9-phenyl-2,7-difluorofluorenone, 1-(2-chlorotrityl)-3,5-dimethylpyrazole, a-[4-(morpholinomethyl)-(2-pyridyl)]benzhydrol, 5-(5-methoxy-2-(N-methylcarbamoyl)-phenyl)dibenzosuberol, N-(2-chlorotrityl)-L-prolinol acetate, 1-(2-chlorotrityl)-2-methylimidazole, 1-(2-chlorotrityl)-1,2,4-triazole, 1, S-bis(4,4′,4″-trichlorotrityl)-1,2,4-triazole-3-thiol, N-((2,6-dichloro-3-methylphenyl)diphenyl)methylimidazole, dynorphin-1,13-diamide, trans-(1R,2R)-3,4-dichloro-N-methyl-N-[2-(1-pyrrolidinyl)cyclohexyl]benzeneacetamide, bremazocine, ethylketocyclazocine, and naloxone.
